(19) United States
(12) Patent Application Publication
(10) Pub. No.: US 2026/0144536 A1
(43) Pub. Date: May 28, 2026

(54) Suture Cutting Device

(71) Applicant: Mavation, LLC, Las Vegas, NV (US)

(72) Inventors: Rusty Allen, San Diego, CA (US); Magda MORRISON, San Diego, CA (US)

(21) Appl. No.: 19/399,782

(22) Filed: Nov. 25, 2025

Related U.S. Application Data
(60) Provisional application No. 63/725,898, filed on Nov. 27, 2024.

(51) Int. Cl. A61B 17/04; A61B 17/06; A61B 90/00

(52) U.S. Cl. CPC A61B 17/0467; A61B 17/06161; A61B 90/08; A61B 2090/0805

(57) ABSTRACT

Systems and methods are provided for a cutting device comprising a base portion configured to rest on a surface. A top portion is connected to the base portion. The base portion and the top portion form a gap adjacent to the base portion and the top portion. A blade extends between the base portion and the top portion and is configured to cut suture traversed through the gap. The base portion and the top portion and configured to prevent access to the blade of objects having more than a predetermined width and to allow access to the blade of objects having less than a predetermined width.

### CROSS-REFERENCE TO RELATED APPLICATIONS

This application claims priority to U.S. Provisional Application No. 63/725,898, filed Nov. 27, 2024, which is incorporated herein by reference in its entirety.

### FIELD

The present disclosure relates to cutting devices, and more particularly to medical cutting devices for cutting suture from medical needles.

### BACKGROUND

In medical procedures, a suture is often used to hold together edges of a wound or surgical incision. A suture may be attached to a suture needle that is used to apply the suture to the wound or surgical incision. A needle driver may be used to guide the suture needle during medical operations. To account for suture needles and other needles (e.g., sharps) used in a medical operation, medical personnel may place suture needles with attached suture in a needle counter or surgical needle counting device. Medical personnel may trim some suture from the suture needle prior to placement in the needle counter or surgical needle counting device for safety or organizational purposes. In some examples, users may hold a needle driver with a suture needle and attached suture in one hand, while using scissors in another hand to trim suture from the suture needle. Such examples may be burdensome, dangerous, and result in suture needles with inconsistent lengths of attached suture.

### SUMMARY

Systems and methods are provided for a cutting device comprising a base portion configured to rest on a surface. A top portion is connected to the base portion. The base portion and the top portion form a gap adjacent to the base portion and the top portion. A blade extends between the base portion and the top portion and is configured to cut suture traversed through the gap. The base portion and the top portion and configured to prevent access to the blade of objects having more than a predetermined width and to allow access to the blade of objects having less than a predetermined width.

As another example, a surgical needle counting device comprises a suture cutting device including a base portion, a top portion, and a blade. The top portion is coupled to the base portion. The base portion and the top portion form a gap. The blade extends through the gap and is configured to cut suture from a needle to a predetermined length when the suture is traversed through the gap. The surgical needle counting device further comprises a collecting enclosure configured to receive the needle and a counting apparatus having a sensor configured for determining a number of needles in the collecting enclosure.

As another example, a method of cutting suture includes securing a base portion to a lower surface. A top portion is secured to the base portion. The base portion and the top portion form a gap. A blade extends through the gap. Suture is traversed through the gap. The suture is cut with the blade.

### BRIEF DESCRIPTION OF THE DRAWINGS

FIG. 1 depicts a top view of a user operating a suture cutting device, in accordance with some embodiments.

FIG. 2 depicts an isometric view of a suture cutting device, in accordance with some embodiments.

FIG. 3 depicts a side view of a suture cutting device, in accordance with some embodiments.

FIG. 4 depicts a top view of a suture cutting device, in accordance with some embodiments.

FIG. 5 depicts a surgical needle counting device with a suture cutting device, in accordance with some embodiments.

FIG. 6A depicts a side view of a user operating a suture cutting device, in accordance with some embodiments.

FIG. 6B depicts a side view of a user operating a suture cutting device, in accordance with some embodiments.

FIG. 7 depicts a method of cutting suture, in accordance with some embodiments.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

It is to be understood that the phraseology and terminology employed herein are for the purpose of description and should not be regarded as limiting. For example, the use of a singular term, such as, “a” is not intended as limiting of the number of items. Also the use of relational terms, such as but not limited to, “top,” “bottom,” “left,” “right,” “upper,” “lower,” “down,” “up,” “side,” are used in the description for clarity and are not intended to limit the scope of the invention or the appended claims. Further, it should be understood that any one of the features can be used separately or in combination with other features. Other systems, methods, features, and advantages of the invention will be or become apparent to one with skill in the art upon examination of the detailed description. It is intended that all such additional systems, methods, features, and advantages be included within this description, be within the scope of the present invention, and be protected by the accompanying claims.

Suture needles and sutures are often used during surgical operations. It is important to account for these sutures and suture needles during a surgical operation, as they can pose risks to medical staff and patients if unattended or unaccounted for. For example, if an unaccounted for suture needle is retained in a person's body at the end of a surgical procedure, there is a high risk of infections, tissue damage, or other adverse effects. To account for suture and suture needles, medical personnel may use a device to keep track of several different needles that are used during a surgical operation. For example, medical personnel may place used suture needles with attached suture in a needle counter box or a surgical needle counting device. An example surgical needle counting device is described in U.S. Patent Application Publication No. 2022/0387124, published Dec. 8, 2022, which is incorporated herein by reference in its entirety.

At the end of a suture maneuver by a surgeon, the suture thread is typically cut before counting and disposal of the suture needle. Suture cutting using scissors or other cutting implements may result in inconsistent lengths of suture being attached to the suture needle. Undesirably long suture or inconsistent suture can interfere with accurate counting and disposal of suture needles. For example, long suture may prevent a suture needle from properly sitting in a cell of a suture counting box. Irregular suture lengths may also interfere with automated suture counting devices, such as those using optical, inductive, or other counting techniques. Long sutures may also entangle with other sutures or suture needles, creating a “rat's nest” that interferes with accurate counting.

Thus, it may be appropriate to cut (e.g., trim) some excess suture from a suture needle before placing the suture and suture needles in the needle counter box or the surgical needle counting device. Removing some excess suture can facilitate easier counting and categorization of the suture needles. In some examples, medical personnel, such as a surgical scrub nurse, may hold a needle driver with a suture needle and attached suture in one hand, and use another hand to trim some excess suture from the suture needle with a cutting device (e.g., scissors).

Systems and methods disclosed herein include a suture cutting device configured to cut suture from suture needles to consistent lengths, limiting the need for operating external cutting devices with one's hands. The suture cutting device may provide the reliable cutting of suture, while ensuring safety to users. Systems and methods disclosed herein may also allow simple suture cutting for both left-and right-handed individuals. The suture cutting device may be mountable or securable on multiple platforms to increase convenience for users.

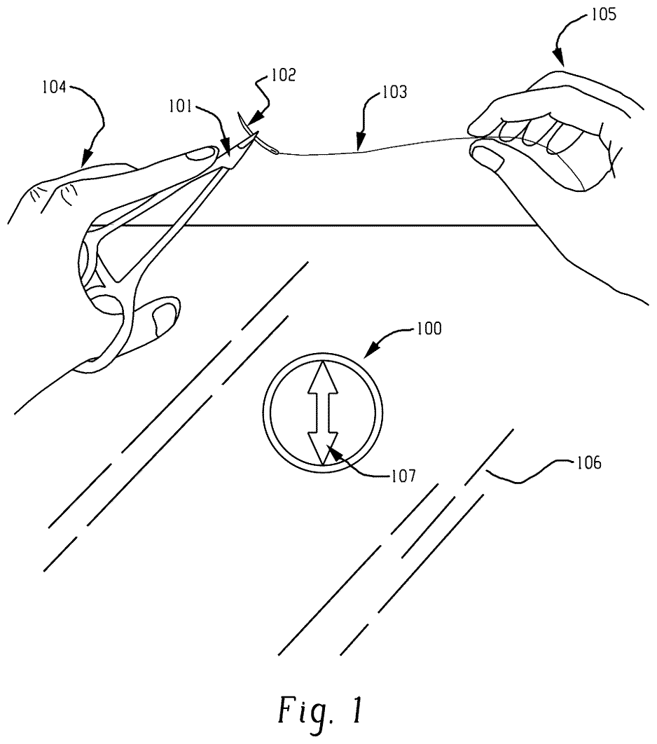

FIG. 1 depicts a top view of a user operating a suture cutting device, in accordance with some embodiments. In the example shown in FIG. 1, the suture cutting device 100 is attached (e.g., mounted) to a lower surface 106. The lower surface 106 may be located on a mayo stand, a surgical needle counting device, or another solid surface. The user holds a needle driver 101 in a first hand (e.g., a left hand). The needle driver 101 holds a suture needle 102. Suture 103 is attached to the suture needle 102. The needle driver 101, suture needle 102, and suture 103 may have been previously used, for example, in a medical operation such as a suturing or a surgery. The user holds the suture 103 with a second hand (e.g., a right hand). The user may allow a length of suture 103 between an end of suture needle 102 and the second hand 105, to allow room to pull the suture 103 through the suture cutting device 100.

The user may pull the suture 103, together with the needle driver 101 and the suture needle 102, through the suture cutting device 100. The suture cutting device 100 may include a label 107 indicating to the user a direction in which to pull the suture 103. The label 107 may be, for example, an arrow that points perpendicular to a blade (e.g., a one- or two-sided scalpel) positioned below the label 107. As shown in FIG. 1, the label 107 includes arrows pointing in two differing and opposite directions. Thus, the label 107 indicates to the user that the suture 103 may be pulled through the suture cutting device 100 in the direction of either arrow. When the user pulls the suture 103 through the suture cutting device 100, the suture cutting device 100 cuts the suture 103 to a predetermined length on the suture needle 102, as described below. The user can then place the suture needle 102 and attached suture 103 in a needle counter box or a surgical needle counting device.

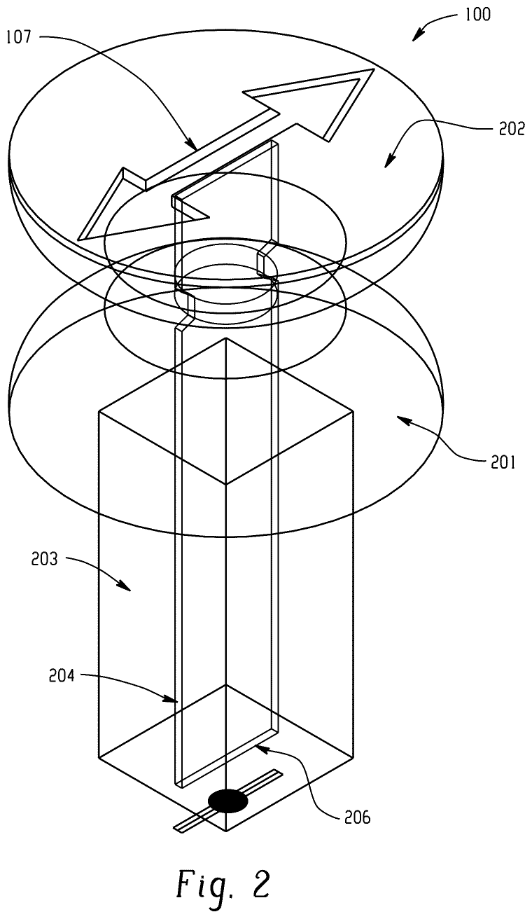

FIG. 2 depicts an isometric view of a suture cutting device, in accordance with some embodiments. In the example shown in FIG. 2, the suture cutting device 100 includes a base portion 201. The base portion 201 may be substantially hemispherical-shaped. For example, the base portion 201 may form a top half of a sphere. The suture cutting device 100 may further include a top portion 202 connected to the base portion 201. The top portion 202 may also be substantially hemispherical-shaped. In the example shown in FIG. 2, the top portion 202 forms a bottom half of a sphere. A bottom of the top portion 202 may be connected to, for example, a top of the base portion 201. The top portion 202 may include a flat upper surface. The upper surface may include the label 107. As described above, the label 107 may be, for example, an arrow that indicates to a user a direction in which to pull suture through the suture cutting device 100.

The suture cutting device 100 may further include a mounting portion 203. The mounting portion 203 is connected to a lower side of the base portion 201. In the example shown in FIG. 2, the mounting portion 203 is a shape of a rectangular prism. However, the mounting portion 203 may be a different shape in other example embodiments. The mounting portion 203 may be configured to be inserted into an aperture in the mayo stand or the surgical needle counting device. The aperture in the mayo stand or the surgical needle counting device may be shaped with substantial similarity to a lateral cross-section of the mounting portion. For example, an aperture holding the mounting portion of the suture cutting device 100 depicted in FIG. 2 may be a square shape. The mounting portion 203 may provide stability to the suture cutting device 100 during operation. For example, when the suture is pulled through the suture cutting device 100, a lateral force may be exerted on the suture cutting device 100. The mounting portion 203 may ensure that the force does not move the suture cutting device 100.

A slot 206 may extend through the mounting portion 203, the base portion 201, and the top portion 201. The slot may extend across all or a portion of a length of the mounting portion. A blade 204 may be removably inserted in the slot 206. For example, the blade 204 may be inserted and removed from the suture cutting device 100 at the slot 206 at the bottom of the mounting portion 203. The blade 204 may be, for example, a No. 11 surgical blade.

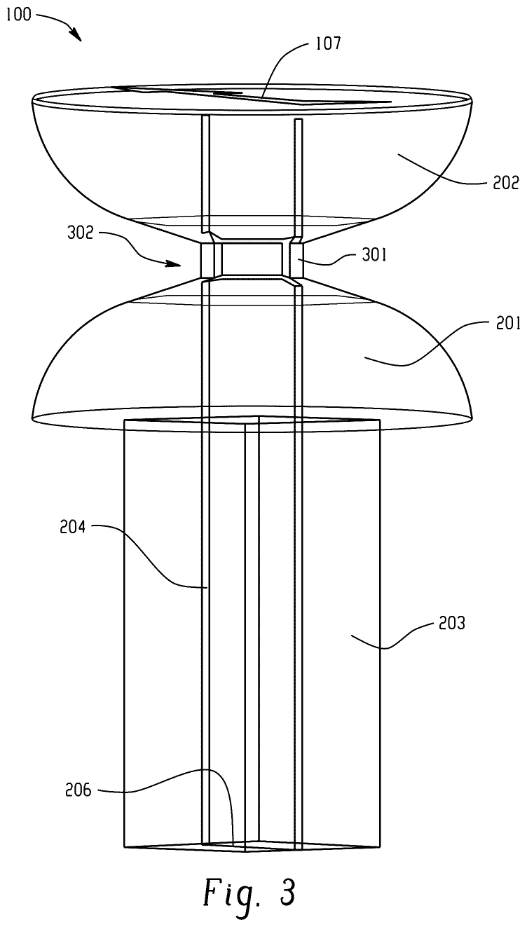

FIG. 3 depicts a side view of a suture cutting device, in accordance with some embodiments. As shown in FIG. 3, the base portion 201 and the top portion 202 form a gap 302. The gap 302 may become more narrow from an outer area to a center of the suture cutting device 100. The top portion 202 and the base portion 201 include openings where they connect with each other. The openings at the connection of the base portion 201 and the top portion 202 may allow exposed portions 301 of the blade 204 to extend into the gap 302. The exposed portions 301 may be based on the label 107 on the upper surface of the top portion 202. For example, the exposed portions 301 may be aligned with each end of the arrow on the upper surface of the top portion 202. The exposed portions 301 of the blade 204 may cut the suture 103 as it passes through the suture cutting device 100.

A part of the top portion 202 and the base portion 201 may extend on each side of exposed portion 301 of the blade 204. For example, a predetermined width of the base portion 201 and the top portion 202 may extend on a left side and a right side of the exposed portion 301 of the blade 204. During operation, the user may bring the suture needle 102 in close proximity or touching the part of the top portion 202 and base portion 201 extending on the left or right side of the exposed portion 301 of the blade 204. Accordingly, the user can ensure that the suture 103 is cut to substantially the predetermined length from the end of the suture needle 102.

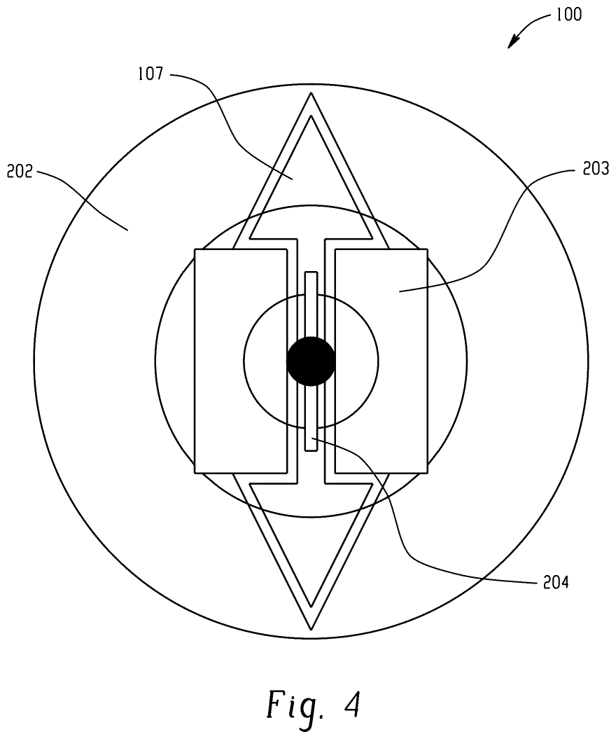

FIG. 4 depicts a top view of a suture cutting device, in accordance with some embodiments. As shown in FIG. 4, the top of the suture cutting device 100 may be substantially circular. As shown in FIG. 4, the mounting portion 203 may be located in substantially the center of the circular upper surface of the top portion 202. Furthermore, the slot 206 may be located in substantially the center of the mounting portion 203.

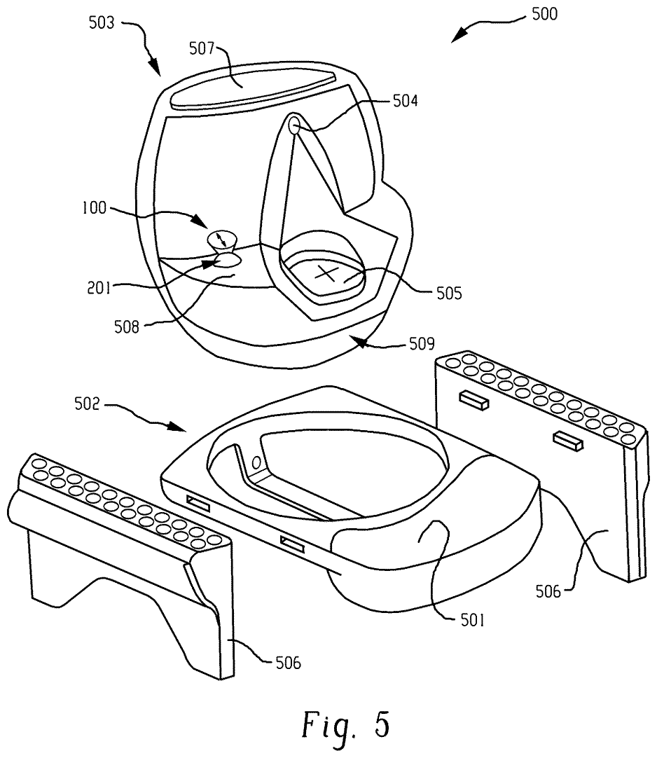

FIG. 5 depicts a surgical needle counting device with a suture cutting device, in accordance with some embodiments. As described above, the surgical needle counting device 500 may be described in U.S. Patent Application Publication No. 2022/0387124, published Dec. 8, 2022, which is incorporated herein by reference in its entirety. The surgical needle counting device 500 may include a vision system for determining that an object, such as surgical needles or sharps, has been received. In embodiments, the vision system may be used to identify characteristics of the received surgical needle or sharp (e.g., size, shape, and type). In some examples, the surgical needle counting device 500 may include a surgical needle management software application that includes a model trained using a machine learning technique. The machine learning technique, for example, may process large numbers (e.g., hundreds, thousands, millions, billions) of needle images in which each image is associated with metadata, such as a size, shape, material, needle type, etc. associated with the needle. The software application's model (e.g., a neural network model) may be trained based on those tagged images so as to recognize those characteristics of future needles captured via its vision system (e.g., via a camera in the surgical needle counting device 500).

In the example depicted in FIG. 2, the surgical needle counting device 500 includes a counting apparatus 503 that may be formed from a housing that is configured to sit within a support structure 502. The support structure 502 may be connected to an external structure (not shown) such as an arm extending from a wheeled vertical stand. The support structure 502 includes a recess for the counting apparatus 503 to rest within during operation as well as a disposal area 501 for depositing waste (e.g., suture material removed from the end of a needle). One or more sharps caddies 506 may be attached to the support structure 502 for depositing and accounting for certain needles and sharps that are not an appropriate size for depositing into the counting apparatus 503. The sharps caddies 506 may be formed of hard plastic to prevent penetration by sharps deposited sharp end down into the sharps caddies 506. The counting apparatus 503 may include sensor equipment 507. The sensor equipment 507 may be enclosed within the counting apparatus 503 to not contaminate the surgical field, even if the sensor equipment 507 is not sterile.

The suture cutting device 100 may be connected to a surface 508 of the counting apparatus 503. For example, the mounting portion 203 (FIG. 2) may be inserted into an opening (not shown) within the surface 508 of the counting apparatus 503. Alternatively, the suture cutting device 100 may not include the mounting portion 203 and the base portion 201 may be removably or securely attached to the surface 508. As described above with reference to FIG. 1, the user may hold the needle driver 101 together with the suture needle 102 in one hand and the suture 103 in another hand, and pull the suture through the suture cutting device 100 while it is mounted to the surface 508. The suture cutting device 100 may cut excess suture 103 from the suture needle 102 such that a predetermined length of suture 103 remains on the suture needle 102. The user may then place the suture needle 102 on top of a target pad 505. During a surgical operation, a needle may be placed by a surgeon or operating room technician onto a target pad 505. The counting apparatus 503 may detect the presence of a needle on the target pad 505 automatically, such as via a pressure sensor, an inductive sensor, or via a camera. A camera used to detect a needle may be present, for example, near a light source 504 for use in darkened operating rooms. The presence of a needle on the target pad 505 may also be manually indicated to the counting apparatus 503, such as by a button press, a voice command, or gesture.

When a needle is deemed present on the target pad 505, a mechanical mechanism rotates the target pad 505 to a back side of the counting apparatus 503, under the sensor equipment 507. Sensor equipment 507 may increment a surgical needle count as appropriate. A second target pad may then be rotated to the front of the counting apparatus 503, to the position depicted in FIG. 2. A magnet (not shown) under the first target pad 505 may be energized to aid in holding the needle on the first target pad 505. The first target pad may now be positioned in a light controlled environment (e.g., a tube into which polarized light is projected while shielding the controlled environment from outside light). A camera (e.g., a camera directed at the controlled environment, focused based on a known reference printed on the target, such as microdots) may take one or more images of the needle on the first pad. The images may be used for recognition of the presence of the needle, characteristics of the needle, or the type of needle (e.g., by comparing characteristics of the needle to those of known needle types in a database, by image recognition provided by a machine learning-trained model).

When a subsequent needle is confirmed on the second target pad, the first target pad 505 and the second target pad may again be rotated such that the first target pad 505 returns to the front position for receipt of another needle. In that rotation process from the controlled image capture environment, the needle may be scraped, wiped, or otherwise removed from the first target pad 505 such that the needle falls into its collection enclosure 509 at the bottom of the counting apparatus 503 for subsequent disposal. During the removal of the needle from the first target pad 505, the magnet under the first target pad 505 may be deenergized to aid in removal. The entire collection enclosure 509 or the entire housing of the counting apparatus 503 may be used to contain needles and sharps.

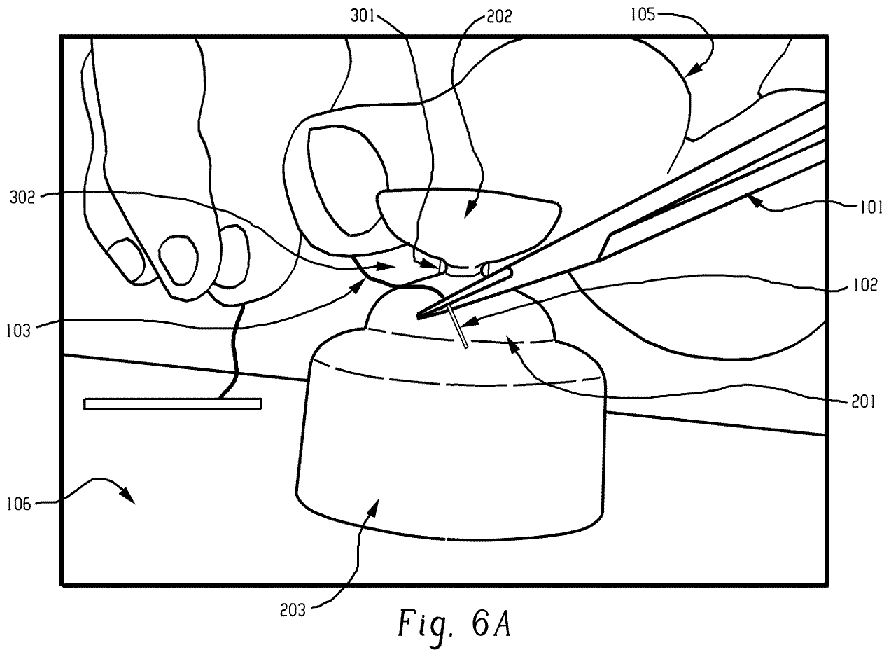

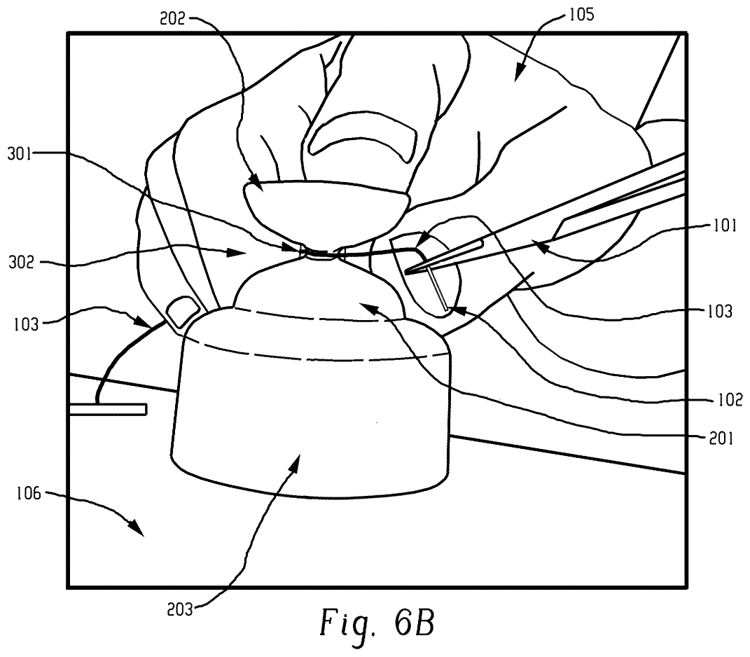

FIGS. 6A and 6B depict side views of a user operating a suture cutting device, in accordance with some embodiments. As shown in FIG. 6A, the suture cutting device 100 may include a mounting portion 203 that is as large or larger than the base portion 201. Furthermore, the mounting portion 203 may have a cylindrical shape, or may have a different shape. The mounting portion 203 is attached to the lower surface 106. In the example shown in FIG. 6A, the user holds the needle driver 101 in the first hand 104 (not shown). The needle driver 101 holds the suture needle 102. The user holds the suture 103 attached to the suture needle 102 in the second hand 105. The user extends their first hand 104 and second hand 105 away from their body past the suture cutting device 100 such that the suture 103 extends laterally on the side of the suture cutting device 100 opposite the user.

As shown in FIG. 6B, the user then pulls the suture needle 102 and attached suture 103 through the gap 302. The gap prevents objects greater than a predetermined width (e.g., the user's fingers) from accessing the exposed portion 301 of the blade within the gap. The user may allow the suture needle 102 to touch, or come within close proximity of, the base portion 201 and the top portion 202 to allow the exposed portion 301 of the blade to cut the suture 103 to a predetermined length, as described above. After the user has cut the excess suture 103 from the suture needle 102, the user can place the suture needle 102 and remaining suture in 103 in the needle counter box or surgical needle counting device, and discard the excess suture 103.

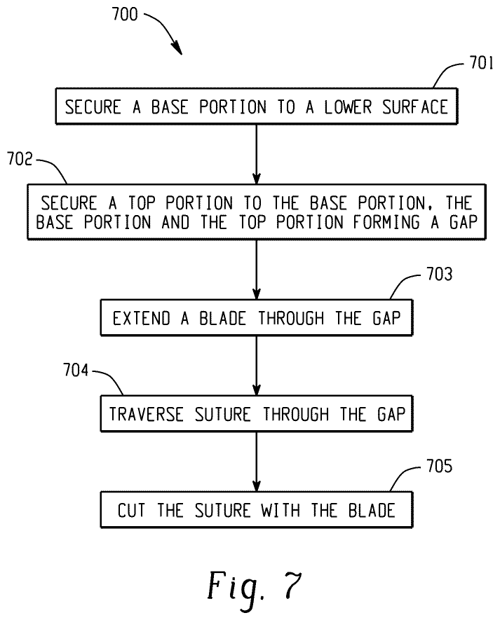

FIG. 7 depicts a method of cutting suture, in accordance with some embodiments. In the example shown in FIG. 7, the method 700 includes a first step 701 of securing a base portion to a lower surface. At 702, a top portion is secured to the base portion. The base portion and the top portion may form a gap. At 703, a blade is extended through the gap. At 704, suture is traversed through the gap. At 705, suture is cut with the blade.

It will be appreciated by those skilled in the art that changes could be made to the embodiments described above without departing from the broad inventive concept thereof. It is understood, therefore, that the invention disclosed herein is not limited to the particular embodiments disclosed, and is intended to cover modifications within the spirit and scope of the present invention.

What is claimed is:

1. A cutting device comprising:
a base portion configured to rest on a surface;
a top portion connected to the base portion, the base portion and the top portion forming a gap adjacent to the base portion and the top portion; and
a blade extending between the base portion and the top portion, the blade configured to cut suture traversed through the gap, the base portion and the top portion configured to prevent access to the blade of objects having more than a predetermined width and to allow access to the blade of objects having less than a predetermined width.

2. The cutting device of claim 1, wherein the base portion and the top portion are substantially dome-shaped.

3. The cutting device of claim 1, wherein the predetermined width is substantially a width of a human finger.

4. The cutting device of claim 1, further comprising a mounting portion coupled to the base portion, the mounting portion configured to secure the cutting device to the surface.

5. The cutting device of claim 4, wherein the surface is a surface of a surgical needle counting device.

6. The cutting device of claim 1, wherein the surface is a surface of a mayo stand.

7. The cutting device of claim 1, wherein the blade is a No. 11 surgical blade.

8. The cutting device of claim 1, further comprising a label indicating a direction for a user to pull the suture.

9. The cutting device of claim 8, wherein the label is on the top portion.

10. The cutting device of claim 8, wherein the label is an arrow.

11. The cutting device of claim 1, wherein the blade extends through the base portion and the top portion.

12. The cutting device of claim 11, further comprising a slot extending through the base portion and the top portion, the slot configured to receive the blade.

13. A surgical needle counting device comprising:
a suture cutting device including:
a base portion;
a top portion coupled to the base portion, the base portion and the top portion forming a gap; and
a blade extending through the gap, the blade configured to cut suture from a needle to a predetermined length when the suture is traversed through the gap;
a collecting enclosure configured to receive the needle; and
a counting apparatus having a sensor configured for determining a number of needles in the collecting enclosure.

14. The surgical needle counting device of claim 13, the suture cutting device further comprising a mounting portion coupled to the base portion, the mounting portion configured to be received at a recess within the surgical needle counting device.

15. The surgical needle counting device of claim 14, further comprising a slot extending through the mounting portion, the base portion, and the top portion, the slot configured to receive the blade.

16. The surgical needle counting device of claim 13, the suture cutting device further comprising a label indicating a direction for a user to pull the suture.

17. The surgical needle counting device of claim 13, wherein the base portion and the top portion are configured to prevent access to the blade of objects having more than a predetermined width and to allow access to the blade of objects having less than a predetermined width.

18. The surgical needle counting device of claim 13, wherein the base portion and the top portion are substantially dome-shaped.

19. A method of cutting suture comprising:
securing a base portion to a lower surface;
securing a top portion to the base portion, the base portion and the top portion forming a gap;
extending a blade through the gap;
traversing suture through the gap; and
cutting the suture with the blade.

20. The method of claim 19, further comprising preventing access to the blade of objects having more than a predetermined width.
